(19) United States
(12) Patent Application Publication
(10) Pub. No.: US 2009/0264502 A1
(43) Pub. Date: Oct. 22, 2009

(54) COMPOSITIONS AND THEIR USES DIRECTED TO HSP27

(76) Inventors: C. Frank Bennett, Carlsbad, CA (US); Nicholas M. Dean, Olivenhain, CA (US); Kenneth W. Dobie, Del Mar, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/064,330

(22) PCT Filed: Aug. 24, 2006
(86) PCT No.: PCT/US06/33407

§ 371 Date: Aug. 18, 2008

Related U.S. Application Data
(60) Provisional application No. 60/711,401, filed on Aug. 25, 2005.

(51) Int. Cl. A61K 31/7125; C07H 21/02; A61P 35/00

(52) U.S. Cl. 514/44 A; 536/23.1

(57) ABSTRACT

Disclosed herein are compounds, compositions and methods for modulating the expression of HSP27 in a cell, tissue or animal. Also provided are uses of disclosed compounds and compositions in the manufacture of a medicament for treatment of diseases and disorders.

### RELATED APPLICATIONS

This application claims the benefit of priority of U.S. Provisional Application Ser. No. 60/711,401, filed Aug. 25, 2005, which is herein incorporated by reference in its entirety.

### INCORPORATION OF SEQUENCE LISTING

A copy of the sequence listing in both a paper form and a computer-readable form is provided herewith and incorporated by reference. The computer-readable form is provided on a diskette containing the file named BIOL0076WOSEQ.txt, which was created on Aug. 23, 2006.

### FIELD OF THE INVENTION

Disclosed herein are compounds, compositions and methods for modulating the expression of HSP27 in a cell, tissue or animal.

### BACKGROUND

Heat shock proteins (HSPs) comprise several different families of proteins that are induced in response to a wide variety of physiological and environmental insults, such as heat shock, reactive oxygen species, ischemia, trauma and disease. One such protein which is highly induced during the stress response is HSP27, a 27-kDa protein. Expression of HSP27 correlates with increased survival in response to cytotoxic stimuli and has been shown to prevent cell death by a wide variety of agents that cause apoptosis. HSP27 is a molecular chaperone with an ability to interact with a large number of proteins. Recent evidence has shown that HSP27 regulates apoptosis through an ability to interact with key components of the apoptotic signaling pathway, in particular, those involved in caspase activation and apoptosis (Concannon et al. (2003) Apoptosis 8(I):61-70).

The role of HSP27 in regulating cell death suggests HSP27 may contribute to the development of hyperproliferative disorders, such as cancer. Accordingly, overexpression of heat shock proteins can increase the tumorigenic potential of tumor cells, further suggesting that HSP27 may participate in oncogenesis (Garrido et al. (2003) Cell Cycle 2(6):579-84).

U.S. Pre-Grant Publication No. 2004-0127441 and PCT Publication WO 2004/030660 disclose antisense oligonucleotides and RNAi nucleotides with sequence specificity for HSP27. Use of the antisense oligonucleotides and RNAi nucleotides as therapeutic agents for treatment of prostate cancer is discussed.

Antisense technology is an effective means for reducing the expression of one or more specific gene products and is uniquely useful in a number of therapeutic, diagnostic, and research applications. Provided herein are antisense compounds for use in modulation of HSP27 expression.

### SUMMARY

Provided herein are antisense compounds targeted to a nucleic acid encoding HSP27. Further provided are methods of modulating the expression of HSP27 in cells, tissues or animals comprising contacting said cells, tissues or animals with one or more of the compounds or compositions provided herein. Pharmaceutical, therapeutic and other compositions comprising the compounds described herein are also provided.

In one embodiment, the antisense compounds are targeted to nucleotides 618 to 660 of a nucleic acid molecule encoding human HSP27 (SEQ ID NO: 2). In another embodiment, the antisense compounds are targeted to nucleotides 229 to 248 of a nucleic acid molecule encoding human HSP27 (SEQ ID NO: 2). In another embodiment, the antisense compounds are targeted to nucleotides 697 to 716 of a nucleic acid molecule encoding human HSP27 (SEQ ID NO: 2). In another embodiment, the antisense compounds are targeted to nucleotides 464 to 534 of a nucleic acid molecule encoding human HSP27 (SEQ ID NO: 2). Antisense compounds targeting smaller regions with the specified nucleotide ranges also are contemplated.

In some embodiments, the antisense compounds provided herein are modified. Such modifications include internucleoside, sugar and nucleobase modifications. In one embodiment, antisense compounds comprise at least one phosphorothioate internucleoside linkage. In another embodiment, antisense compounds comprise at least one 2′-O-methoxyethyl sugar moiety. In yet another embodiment, antisense compounds comprise at least one 5-methylcytosine residue.

Also provided is the use of the compounds or compositions in the manufacture of a medicament for the treatment of one or more conditions associated with HSP27. Further contemplated are methods where cells or tissues are contacted in vivo with an effective amount of one or more of the compounds or compositions. Also provided are ex vivo methods of treatment that include contacting cells or tissues with an effective amount of one or more of the compounds or compositions described herein and then introducing said cells or tissues into an animal.

### DETAILED DESCRIPTION

### Overview

Antisense technology is an effective means for reducing the expression of one or more specific gene products and is uniquely useful in a number of therapeutic, diagnostic, and research applications. Provided herein are antisense compounds useful for modulating gene expression and associated pathways via antisense mechanisms of action based on target degradation or target occupancy.

The principle behind antisense technology is that an antisense compound, which hybridizes to a target nucleic acid, modulates gene expression activities such as transcription, splicing or translation through one of a number of antisense mechanisms. The sequence specificity of antisense compounds makes them extremely attractive as tools for target validation and gene functionalization, as well as therapeutics to selectively modulate the expression of genes involved in disease.

HSP27 is known to influence cell proliferation and apoptosis, processes which are often disregulated in a number of disease states such as cancer. Thus, there is a great need for the development of specific HSP27 inhibitors. Disclosed herein are oligomeric compounds, including antisense oligonucleotides and other antisense compounds for use in modulating the expression of nucleic acid molecules encoding HSP27. This is accomplished by providing oligomeric compounds which hybridize with one or more target nucleic acid molecules encoding HSP27.

As used herein, “targeting” or “targeted to” refer to the process of designing an oligomeric compound such that the compound specifically hybridizes with a selected nucleic acid molecule, or a particular region of thereof. In the context of the instant disclosure, oligomeric compounds targeted to a particular range of nucleotides specifically hybridize within, but not outside of, the specified region.

As used herein, “hybridization” means the pairing of complementary strands of oligomeric compounds. An oligomeric compound is specifically hybridizable when there is a sufficient degree of complementarity to avoid non-specific binding of the oligomeric compound to non-target nucleic acid sequences.

As used herein, “antisense mechanisms” are all those involving hybridization of a compound with target nucleic acid, wherein the outcome or effect of the hybridization is either target degradation or target occupancy with concomitant stalling of the cellular machinery involving, for example, transcription or splicing.

### Target Nucleic Acids

Targeting an oligomeric compound to a particular target nucleic acid molecule can be a multistep process. The process usually begins with the identification of a target nucleic acid whose expression is to be modulated. As used herein, the terms “target nucleic acid” and “nucleic acid encoding HSP27” encompass DNA encoding HSP27, RNA (including pre-mRNA and mRNA) transcribed from such DNA, and also cDNA derived from such RNA.

The targeting process usually also includes determination of at least one target region, segment, or site within the target nucleic acid for the antisense interaction to occur such that the desired effect will result. “Region” is defined as a portion of the target nucleic acid having at least one identifiable structure, function, or characteristic. Target regions may include an exon or an intron. Within regions of target nucleic acids are segments. “Segments” are defined as smaller or sub-portions of regions within a target nucleic acid. “Sites,” as used herein, are defined as unique nucleobase positions within a target nucleic acid.

Provided herein are compositions and methods for modulating the expression of HSP27 (also known as HSPB1; heat shock 27 kD protein 1; HSP28; Heat-shock protein beta-1). Listed in Table 1 are GENBANK(D accession numbers of sequences used to design oligomeric compounds targeted to HSP27. Oligomeric compounds include oligomeric compounds which hybridize with one or more target nucleic acid molecules shown in Table 1, as well as oligomeric compounds which hybridize to other nucleic acid molecules encoding HSP27. The oligomeric compounds may target any region, segment, or site of nucleic acid molecules which encode HSP27. Suitable target regions, segments, and sites include, but are not limited to, the 5′UTR, the start codon, the stop codon, the coding region, the 3′UTR, the 5′cap region, introns, exons, intron-exon junctions, exon-intron junctions, and exon-exon junctions.

TABLE 1
Gene Target Names and Sequences

| Target Name | Species | Genbank ® # | SEQ ID NO |
|---|---|---|---|
| HSP27 | Human | AB020027.1 | 1 |
| HSP27 | Human | NM_001540.2 | 2 |

### Antisense Compounds

The term “oligomeric compound” refers to a polymeric structure capable of hybridizing to a region of a nucleic acid molecule. This term includes oligonucleotides, oligonucleosides, oligonucleotide analogs, oligonucleotide mimetics and chimeric combinations of these. Oligomeric compounds are routinely prepared linearly but can be joined or otherwise prepared to be circular. Moreover, branched structures are known in the art. An “antisense compound” or “antisense oligomeric compound” refers to an oligomeric compound that is at least partially complementary to the region of a nucleic acid molecule to which it hybridizes and which modulates (increases or decreases) its expression. Consequently, while all antisense compounds can be said to be oligomeric compounds, not all oligomeric compounds are antisense compounds. An “antisense oligonucleotide” is an antisense compound that is a nucleic acid-based oligomer. An antisense oligonucleotide can be chemically modified. Nonlimiting examples of oligomeric compounds include primers, probes, antisense compounds, antisense oligonucleotides, external guide sequence (EGS) oligonucleotides and alternate splicers. In one embodiment, the oligomeric compound comprises an antisense strand hybridized to a sense strand. Oligomeric compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound.

The oligomeric compounds provided herein comprise compounds from about 8 to about 80 nucleobases (i.e. from about 8 to about 80 linked nucleosides). One of ordinary skill in the art will appreciate that this comprehends antisense compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the antisense compounds comprise 13 to 50 nucleobases. One having ordinary skill in the art will appreciate that this embodies antisense compounds of 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, or 50 nucleobases.

In one embodiment, the antisense compounds comprise 13 to 30 nucleobases. One having ordinary skill in the art will appreciate that this embodies antisense compounds of 13, 14, 15, 16, 17, 18, 19, 20, 21, 22, 23, 24, 25, 26, 27, 28, 29 or 30 nucleobases.

In some embodiments, the antisense compounds comprise 15 to 30 nucleobases. One having ordinary skill in the art will appreciate that this embodies antisense compounds of 15, 16, 17, 18, 19, 20, 21, 22, 23, 24, 25, 26, 27, 28, 29 or 30 nucleobases.

In one embodiment, the antisense compounds comprise 20 to 30 nucleobases. One having ordinary skill in the art will appreciate that this embodies antisense compounds of 20, 21, 22, 23, 24, 25, 26, 27, 28, 29, or 30 nucleobases.

In one embodiment, the antisense compounds comprise 20 to 24 nucleobases. One having ordinary skill in the art will appreciate that this embodies antisense compounds of 20, 21, 22, 23, or 24 nucleobases.

In one embodiment, the antisense compounds comprise 20 nucleobases.

In one embodiment, the antisense compounds comprise 19 nucleobases.

In one embodiment, the antisense compounds comprise 18 nucleobases.

In one embodiment, the antisense compounds comprise 17 nucleobases.

In one embodiment, the antisense compounds comprise 16 nucleobases.

In one embodiment, the antisense compounds comprise 15 nucleobases.

In one embodiment, the antisense compounds comprise 14 nucleobases.

In one embodiment, the antisense compounds comprise 13 nucleobases.

Antisense compounds 8-80nucleobases in length, or any length therewithin, comprising a stretch of at least eight (8) consecutive nucleobases selected from within the illustrative antisense compounds are considered to be suitable antisense compounds.

Compounds provided herein include oligonucleotide sequences that comprise at least the 8 consecutive nucleobases from the 5′-terminus of one of the illustrative antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Other compounds are represented by oligonucleotide sequences that comprise at least the 8 consecutive nucleobases from the 3′-terminus of one of the illustrative antisense compounds (the remaining nucleobases being a consecutive stretch of the same oligonucleotide beginning immediately downstream of the 340 -terminus of the antisense compound which is specifically hybridizable to the target nucleic acid and continuing until the oligonucleotide contains about 8 to about 80 nucleobases). It is also understood that compounds may be represented by oligonucleotide sequences that comprise at least 8 consecutive nucleobases from an internal portion of the sequence of an illustrative compound, and may extend in either or both directions until the oligonucleotide contains about 8 to about 80 nucleobases.

One having skill in the art armed with the antisense compounds illustrated herein will be able, without undue experimentation, to identify further antisense compounds.

### Hybridization

As used herein, “hybridization” means the pairing of complementary strands of antisense compounds to their target sequence. While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is complementary to the natural nucleobases thymidine and uracil which pair through the formation of hydrogen bonds. The natural base guanine is complementary to the natural bases cytosine and 5-methyl cytosine. Hybridization can occur under varying circumstances.

An antisense compound is specifically hybridizable when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

### Complementarity

“Complementarity,” as used herein, refers to the capacity for precise pairing between two nucleobases on either two oligomeric compound strands or an antisense compound with its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be a complementary position.

“Complementarity” can also be viewed in the context of an antisense compound and its target, rather than in a base by base manner. The antisense compound and the further DNA or RNA are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the antisense compound and a target nucleic acid. One skilled in the art recognizes that the inclusion of mismatches is possible without eliminating the activity of the antisense compound. Compounds provided herein are therefore directed to those antisense compounds that may contain up to about 20% nucleotides that disrupt base pairing of the antisense compound to the target. Preferably the compounds contain no more than about 15%, more preferably not more than about 10%, most preferably not more than 5% or no mismatches. The remaining nucleotides do not disrupt hybridization (e.g., universal bases).

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of the skill in the art is capable of determining an appropriate number of mismatches between oligonucleotides, or between an oligonucleotide and a target nucleic acid, such as by determining melting temperature.

### Identity

Antisense compounds, or a portion thereof, may have a defined percent identity to a SEQ ID NO, or a compound having a specific Isis number. As used herein, a sequence is identical to the sequence disclosed herein if it has the same nucleobase pairing ability. For example, a RNA which contains uracil in place of thymidine in the disclosed sequences would be considered identical as they both pair with adenine. This identity may be over the entire length of the oligomeric compound, or in a portion of the antisense compound (e.g., nucleobases 1-20 of a 27-mer may be compared to a 20-mer to determine percent identity of the oligomeric compound to the SEQ ID NO.) It is understood by those skilled in the art that an antisense compound need not have an identical sequence to those described herein to function similarly to the antisense compound described herein. Shortened versions of antisense compound taught herein, or non-identical versions of the antisense compound taught herein are also contemplated. Non-identical versions are those wherein each base does not have the same pairing activity as the antisense compounds disclosed herein. Bases do not have the same pairing activity by being shorter or having at least one abasic site. Alternatively, a non-identical version can include at least one base replaced with a different base with different pairing activity (e.g., G can be replaced by C, A, or T). Percent identity is calculated according to the number of bases that have identical base pairing corresponding to the SEQ ID NO or antisense compound to which it is being compared. The non-identical bases may be adjacent to each other, dispersed through out the oligonucleotide, or both.

For example, a 16-mer having the same sequence as nucleobases 2-17 of a 20-mer is 80% identical to the 20-mer. Alternatively, a 20-mer containing four nucleobases not identical to the 20-mer is also 80% identical to the 20-mer. A 14-mer having the same sequence as nucleobases 1-14 of an 1 8-mer is 78% identical to the 18-mer. Such calculations are well within the ability of those skilled in the art.

The percent identity is based on the percent of nucleobases in the original sequence present in a portion of the modified sequence. Therefore, a 30 nucleobase antisense compound comprising the full sequence of the complement of a 20 nucleobase active target segment would have a portion of 100% identity with the complement of the 20 nucleobase active target segment, while further comprising an additional 10 nucleobase portion. The complement of an active target segment may constitute a single portion. In a preferred embodiment, the oligonucleotides are at least about 80%, more preferably at least about 85%, even more preferably at least about 90%, most preferably at least 95% identical to at least a portion of the complement of the active target segments presented herein.

It is well known by those skilled in the art that it is possible to increase or decrease the length of an antisense compound and/or introduce mismatch bases without eliminating activity. For example, in Woolf et al. (Proc. Natl. Acad. Sci. USA 89:7305-7309, 1992, incorporated herein by reference), a series of ASOs 13-25 nucleobases in length were tested for their ability to induce cleavage of a target RNA. ASOs 25 nucleobases in length with 8 or 11 mismatch bases near the ends of the ASOs were able to direct specific cleavage of the target mRNA, albeit to a lesser extent than the ASOs that contained no mismatches. Similarly, target specific cleavage was achieved using a 13 nucleobase ASOs, including those with 1 or 3 mismatches. Maher and Dolnick (Nuc. Acid. Res. 16:3341-3358,1988, incorporated herein by reference) tested a series of tandem 14 nucleobase ASOs, and a 28 and 42 nucleobase ASOs comprised of the sequence of two or three of the tandem ASOs, respectively, for their ability to arrest translation of human DHFR in a rabbit reticulocyte assay. Each of the three 14 nucleobase ASOs alone were able to inhibit translation, albeit at a more modest level than the 28 or 42 nucleobase ASOs. It is understood that antisense compounds can vary in length and percent complementarity to the target provided that they maintain the desired activity. Methods to determine desired activity are disclosed herein and well known to those skilled in the art.

### Kits, Research Reagents and Diagnostics

The antisense compounds provided herein can be utilized for diagnostics, and as research reagents and kits. Furthermore, antisense compounds, which are able to inhibit gene expression or modulate gene expression (e.g., modulation of splicing) with specificity, are often used by those of ordinary skill to elucidate the function of particular genes or to distinguish between functions of various members of a biological pathway.

For use in kits and diagnostics, the antisense compounds provided herein, either alone or in combination with other compounds or therapeutics, can be used as tools in differential and/or combinatorial analyses to elucidate expression patterns of a portion or the entire complement of genes expressed within cells and tissues. Methods of gene expression analysis are well known to those skilled in the art.

### Therapeutics

Antisense compounds provided herein can be used to modulate the expression of HSP27 in an animal, such as a human. In one non-limiting embodiment, the methods comprise the step of administering to said animal in need of therapy for a disease or condition associated with HSP27 an effective amount of an antisense compound that modulates expression of HSP27. A disease or condition associated with HSP27 includes, but is not limited to, cancer. Antisense compounds that effectively modulate expression of HSP27 RNA or protein products of expression are considered active antisense compounds.

For example, modulation of expression of HSP27 can be measured in a bodily fluid, which may or may not contain cells; tissue; or organ of the animal. Methods of obtaining samples for analysis, such as body fluids (e.g., sputum, serum), tissues (e.g., biopsy), or organs, and methods of preparation of the samples to allow for analysis are well known to those skilled in the art. Methods for analysis of RNA and protein levels are discussed above and are well known to those skilled in the art. The effects of treatment can be assessed by measuring biomarkers associated with the target gene expression in the aforementioned fluids, tissues or organs, collected from an animal contacted with one or more compounds, by routine clinical methods known in the art. These biomarkers include but are not limited to: liver transaminases, bilirubin, albumin, blood urea nitrogen, creatine and other markers of kidney and liver function; interleukins, tumor necrosis factors, intracellular adhesion molecules, C-reactive protein, chemokines, cytokines, and other markers of inflammation.

The antisense compounds provided herein can be utilized in pharmaceutical compositions by adding an effective amount of a compound to a suitable pharmaceutically acceptable diluent or carrier. Acceptable carriers and diluents are well known to those skilled in the art. Selection of a diluent or carrier is based on a number of factors, including, but not limited to, the solubility of the compound and the route of administration. Such considerations are well understood by those skilled in the art. The compounds provided herein can also be used in the manufacture of a medicament for the treatment of diseases and disorders related to HSP27.

Methods whereby bodily fluids, organs or tissues are contacted with an effective amount of one or more of the antisense compounds or compositions are also contemplated. Bodily fluids, organs or tissues can be contacted with one or more of the compounds described herein resulting in modulation of HSP27 expression in the cells of bodily fluids, organs or tissues. An effective amount can be determined by monitoring the modulatory effect of the antisense compound or compounds or compositions on target nucleic acids or their products by methods routine to the skilled artisan.

Thus, provided herein is the use of an isolated antisense compound targeted to HSP27 in the manufacture of a medicament for the treatment of a disease or disorder by means of the method described above.

### Chemical Modifications

As is known in the art, a nucleoside is a base-sugar combination. The base portion of the nucleoside is normally a heterocyclic base (sometimes referred to as a “nucleobase” or simply a “base”). The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. Within oligonucleotides, the phosphate groups are commonly referred to as forming the internucleoside backbone of the oligonucleotide. The normal linkage or backbone of RNA and DNA is a 3′ to 5′ phosphodiester linkage. It is often preferable to include chemical modifications in oligonucleotides to alter their activity. Chemical modifications can alter oligonucleotide activity by, for example: increasing affinity of an antisense oligonucleotide for its target RNA, increasing nuclease resistance, and/or altering the pharmacokinetics of the oligonucleotide. The use of chemistries that increase the affinity of an oligonucleotide for its target can allow for the use of shorter oligonucleotide compounds.

The term “nucleobase” or “heterocyclic base moiety” as used herein, refers to the heterocyclic base portion of a nucleoside. In general, a nucleobase is any group that contains one or more atom or groups of atoms capable of hydrogen bonding to a base of another nucleic acid. In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable to the compounds described herein. The terms modified nucleobase and nucleobase mimetic can overlap but generally a modified nucleobase refers to a nucleobase that is fairly similar in structure to the parent nucleobase, such as for example a 7-deaza purine, a 5-methyl cytosine, or a G-clamp, whereas a nucleobase mimetic would include more complicated structures, such as for example a tricyclic phenoxazine nucleobase mimetic. Methods for preparation of the above noted modified nucleobases are well known to those skilled in the art.

Antisense compounds provided herein may also contain one or more nucleosides having modified sugar moieties. The furanosyl sugar ring of a nucleoside can be modified in a number of ways including, but not limited to, addition of a substituent group, bridging of two non-geminal ring atoms to form a bicyclic nucleic acid (BNA) and substitution of an atom or group such as —S—, —N(R)— or —C(R₁)(R₂) for the ring oxygen at the 4′-position. Modified sugar moieties are well known and can be used to alter, typically increase, the affinity of the antisense compound for its target and/or increase nuclease resistance. A representative list of preferred modified sugars includes but is not limited to bicyclic modified sugars (BNA's), including LNA and ENA (4′-(CH₂)₂—O-2′ bridge); and substituted sugars, especially 2′-substituted sugars having a 2′-F, 2′-OCH₂ or a 2′-O(CH₂)₂—OCH₃ substituent group. Sugars can also be replaced with sugar mimetic groups among others. Methods for the preparations of modified sugars are well known to those skilled in the art.

The compounds described herein may include internucleoside linking groups that link the nucleosides or otherwise modified monomer units together thereby forming an antisense compound. The two main classes of internucleoside linking groups are defined by the presence or absence of a phosphorus atom. Representative phosphorus containing internucleoside linkages include, but are not limited to, phosphodiesters, phosphotriesters, methylphosphonates, phosphoramidate, and phosphorothioates. Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)2-O—); and N,N′-dimethylhydrazine (—CH₂-N(CH₃)—N(CH₃)—). Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a choral atom can be prepared racemes, choral, or as a mixture. Representative choral internucleoside linkages include, but are not limited to, alkylphosphonates and phosphorothioates. Methods of preparation of phosphorous-containing and non-phosphorous-containing linkages are well known to those skilled in the art.

As used herein the term “mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

As used herein the term “nucleoside” includes, nucleosides, abasic nucleosides, modified nucleosides, and nucleosides having mimetic bases and/or sugar groups.

As used herein, the term “oligonucleotide” refers to an oligomeric compound which is an oligomer or polymer of ribonucleic acid (RNA) or deoxyribonucleic acid (DNA). This term includes oligonucleotides composed of naturally- and non-naturally-occurring nucleobases, sugars and covalent internucleoside linkages, possibly further including non-nucleic acid conjugates.

The present disclosure provides compounds having reactive phosphorus groups useful for forming internucleoside linkages including for example phosphodiester and phosphorothioate internucleoside linkages. Methods of preparation and/or purification of precursors or antisense compounds are not a limitation of the compositions or methods provided herein. Methods for synthesis and purification of DNA, RNA, and the antisense compounds provided herein are well known to those skilled in the art.

AS used herein the term “chimeric antisense compound” refers to an antisense compound, having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified. In general a chimeric oligomeric compound will have modified nucleosides that can be in isolated positions or grouped together in regions that will define a particular motif. Any combination of modifications and or mimetic groups can comprise a chimeric oligomeric compound.

Chimeric oligomeric compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

As used herein, the term “fully modified motif” refers to an antisense compound comprising a contiguous sequence of nucleosides wherein essentially each nucleoside is a sugar modified nucleoside having uniform modification.

The compounds described herein contain one or more asymmetric centers and thus give rise to enantiomers, diastereomers, and other stereoisomeric configurations that may be defined, in terms of absolute stereochemistry, as (R) or (S), α or β, or as (D) or (L) such as for amino acids et al. The present disclosure is meant to include all such possible isomers, as well as their racemic and optically pure forms.

In one aspect, antisense compounds are modified by covalent attachment of one or more conjugate groups. Conjugate groups may be attached by reversible or irreversible attachments. Conjugate groups may be attached directly to antisense compounds or by use of a linker. Linkers may be mono- or bifunctional linkers. Such attachment methods and linkers are well known to those skilled in the art. In general, conjugate groups are attached to antisense compounds to modify one or more properties. Such considerations are well known to those skilled in the art.

### Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Antisense compounds can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The disclosure is not limited by the method of antisense compound synthesis.

### Oligomer Purification and Analysis

Methods of oligonucleotide purification and analysis are known to those skilled in the art. Analysis methods include capillary electrophoresis (CE) and electrospray-mass spectroscopy. Such synthesis and analysis methods can be performed in multi-well plates. The methods described herein are not limited by the method of oligomer purification.

### Salts, Prodrugs and Bioequivalents

The antisense compounds described herein comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the disclosure is also drawn to prodrugs and pharmaceutically acceptable salts of the antisense compounds, pharmaceutically acceptable salts of such prodrugs, and other bioequivalents.

The term “prodrug” indicates a therapeutic agent that is prepared in an inactive or less active form that is converted to an active form (i.e., drug) within the body or cells thereof by the action of endogenous enzymes, chemicals, and/or conditions. In particular, prodrug versions of the oligonucleotides are prepared as SATE ((S-acetyl-2-thioethyl) phosphate) derivatives according to the methods disclosed in WO 93/24510 or WO 94/26764. Prodrugs can also include antisense compounds wherein one or both ends comprise nucleobases that are cleaved (e.g., by incorporating phosphodiester backbone linkages at the ends) to produce the active compound.

The term “pharmaceutically acceptable salts” refers to physiologically and pharmaceutically acceptable salts of the compounds: i.e., salts that retain the desired biological activity of the parent compound and do not impart undesired toxicological effects thereto. Sodium salts of antisense oligonucleotides are useful and are well accepted for therapeutic administration to humans. In another embodiment, sodium salts of dsRNA compounds are also provided.

### Formulations

The antisense compounds described herein may also be admixed, encapsulated, conjugated or otherwise associated with other molecules, molecule structures or mixtures of compounds.

The present disclosure also includes pharmaceutical compositions and formulations which include the antisense compounds described herein. The pharmaceutical compositions may be administered in a number of ways depending upon whether local or systemic treatment is desired and upon the area to be treated. In a preferred embodiment, administration is topical to the surface of the respiratory tract, particularly pulmonary, e.g., by nebulization, inhalation, or insufflation of powders or aerosols, by mouth and/or nose.

The pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery). In a preferred embodiment, the pharmaceutical formulations are prepared for pulmonary administration in an appropriate solvent, e.g., water or normal saline, possibly in a sterile formulation, with carriers or other agents to allow for the formation of droplets of the desired diameter for delivery using inhalers, nasal delivery devices, nebulizers, and other devices for pulmonary delivery. Alternatively, the pharmaceutical formulations may be formulated as dry powders for use in dry powder inhalers.

A “pharmaceutical carrier” or “excipient” can be a pharmaceutically acceptable solvent, suspending agent or any other pharmacologically inert vehicle for delivering one or more nucleic acids to an animal and are known in the art. The excipient may be liquid or solid and is selected, with the planned manner of administration in mind, so as to provide for the desired bulk, consistency, etc., when combined with a nucleic acid and the other components of a given pharmaceutical composition.

### Combinations

Compositions provided herein can contain two or more antisense compounds. In another related embodiment, compositions can contain one or more antisense compounds, particularly oligonucleotides, targeted to a first nucleic acid and one or more additional antisense compounds targeted to a second nucleic acid target. Alternatively, compositions provided herein can contain two or more antisense compounds targeted to different regions of the same nucleic acid target. Two or more combined compounds may be used together or sequentially. Compositions can also be combined with other non-antisense compound therapeutic agents.

### Nonlimiting Disclosure and Incorporation by Reference

While certain compounds, compositions and methods provided herein have been described with specificity in accordance with certain embodiments, the following examples serve only to illustrate the compounds of the invention and are not intended to limit the same. Each of the references, GenBank accession numbers, and the like recited in the present application is incorporated herein by reference in its entirety.

### EXAMPLES

### Example 1

### Cell Culture and Treatment with Oligomeric Compounds

The effect of oligomeric compounds on target nucleic acid expression was tested in A549 cells. The human lung carcinoma cell line A549 was obtained from the American Type Culture Collection (Manassas, Va.). A549 cells were routinely cultured in DMEM, high glucose (Invitrogen Life Technologies, Carlsbad, Calif.) supplemented with 10% fetal bovine serum, 100 units per ml penicillin, and 100 micrograms per ml streptomycin (Invitrogen Life Technologies, Carlsbad, Calif.). Cells were routinely passaged by trypsinization and dilution when they reached approximately 90% confluence. Cells were seeded into 96-well plates (Falcon-Primaria #3872) at a density of approximately 5000 cells/well for use in oligomeric compound transfection experiments.

When cells reached 65-75% confluency, they were treated with oligonucleotide. Oligonucleotide was mixed with LIPOFECTIN™ (Invitrogen Life Technologies, Carlsbad, Calif.) in Opti-MEM™-1 reduced serum medium (Invitrogen Life Technologies, Carlsbad, Calif.) to achieve the desired concentration of oligonucleotide and a LIPOFECTIN™ concentration of 2.5 or 3 μg/Ml per 100 nm oligonucleotide. This transfection mixture was incubated at room temperature for approximately 0.5 hours. For cells grown in 96-well plates, wells were washed once with 100 μL OPTI-MEM™-1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

Control oligonucleotides are used to determine the optimal oligomeric compound concentration for a particular cell line. Furthermore, when oligomeric compounds are tested in oligomeric compound screening experiments or phenotypic assays, control oligonucleotides are tested in parallel. The concentration of oligonucleotide used varies from cell line to cell line.

### Example 2

### Real-Time Quantitative PCR Analysis of HSP27 MRNA Levels

Quantitation of HSP27 mRNA levels was accomplished by real-time quantitative PCR using the ABI PRISM™ 7600, 7700, or 7900 Sequence Detection System (PE-Applied Biosystems, Foster City, Calif.) according to manufacturer's instructions.

Prior to quantitative PCR analysis, primer-probe sets specific to the target gene being measured were evaluated for their ability to be “multiplexed” with a GAPDH amplification reaction. After isolation the RNA is subjected to sequential reverse transcriptase (RT) reaction and real-time PCR, both of which are performed in the same well. RT and PCR reagents were obtained from Invitrogen Life Technologies (Carlsbad, Calif.). RT, real-time PCR was carried out in the same by adding 20 ∞L PCR cocktail (2.5× PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5× ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by RT, real-time PCR were normalized using either the expression level of GAPDH, a gene whose expression is constant, or by quantifying total RNA using RiboGreen™ (Molecular Probes, Inc. Eugene, Oreg.). GAPDH expression was quantified by RT, real-time PCR, by being run simultaneously with the target, multiplexing, or separately. Total RNA was quantified using RiboGreen™ RNA quantification reagent (Molecular Probes, Inc. Eugene, Oreg.).

170 μL of RiboGreen™ working reagent (RiboGreen™ reagent diluted 1:350 in 10 mM Tris-HC1, 1 mM EDTA, pH 7.5) was pipetted into a 96-well plate containing 30 μL purified cellular RNA. The plate was read in a CytoFluor 4000 (PE Applied Biosystems) with excitation at 485 nm and emission at 530 nm.

Probes and primers for use in real-time PCR were designed to hybridize to target-specific sequences. The primers and probes and HSP27 target nucleic acid sequences to which they hybridize are presented in Table 2. The target-specific PCR probes have FAM covalently linked to the 5′ end and TAMRA or MGB covalently linked to the 3′ end, where FAM is the fluorescent dye and TAMRA or MGB is the quencher dye.

TABLE 2

HSP27-specific primers and probes for use in real-time PCR

| | | Target | | | |
|---|---|---|---|---|---|
| Target | | SEQ ID | Sequence | | SEQ ID |
| Name | Species | NO | Description | Sequence (5′ to 3′) | NO |
| HSP27 | Human | 2 | Forward Primer | TCCCTGGATGTCAACCACTTC | 3 |
| HSP27 | Human | 2 | Reverse Primer | TCTCCACCACGCCATCCT | 4 |
| HSP27 | Human | 2 | Probe | CCCGGACGAGCTGACGGTCAA | 5 |

### Example 3

### Antisense Inhibition of Gene Targets by Oligomeric Compounds

A series of oligomeric compounds was designed to target different regions of HSP27, using published sequences cited in Table 1. The compounds are shown in Table 3. All compounds in Table 3 are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucleotide. All cytidine residues are 5-methylcytidines. A549 cells were transfected with 100 nM of each compound and the compounds were analyzed for their effect on HSP27 mRNA levels by quantitative real-time PCR as described in other examples herein. Data are averages from experiments in which cultured cells were treated with the disclosed oligomeric compounds. Shown in Table 3 is the SEQ ID NO. of the sequence to which each oligomeric compound is targeted.

A reduction in expression is expressed as percent inhibition. If the target expression level of oligomeric compound-treated cell was higher than control, percent inhibition is expressed as zero inhibition. The target regions to which these oligomeric compounds are inhibitory are herein referred to as “validated target segments.”

TABLE 3

Inhibition of gene target mRNA levels by chimeric oligo-
nucleotides having 2′-MOE wings and deoxy gap

| | Target | | | | |
|---|---|---|---|---|---|
| | SEQ ID | Target | | % | SEQ ID |
| ISIS # | NO | Site | Sequence (5′ to 3′) | Inhibition | NO |
| 178156 | 2 | 7 | GGTATTTTTAGCAGGCGGTG | 0 | 6 |
| 178205 | 2 | 14 | CCAGTGGGGTATTTTTAGCA | 0 | 7 |
| 178181 | 2 | 16 | CTCGAGTCGGGTATTTTTAG | 33 | 8 |
| 178197 | 2 | 21 | TGCTCCTCCAGTCGGGTATT | 6 | 9 |
| 178193 | 2 | 35 | CGGCTGCGCTTTTATGCTCC | 19 | 10 |
| 178159 | 2 | 38 | GCTCGGCTGCGCTTTTATGC | 0 | 11 |
| 178154 | 2 | 39 | GGCTCGGCTGCGCTTTTATG | 2 | 12 |
| 178149 | 2 | 90 | ATGCTGGCTGACTCTGCTCT | 43 | 13 |
| 178157 | 2 | 172 | GAGGCGGCTATGCGGGTACC | 49 | 14 |
| 178184 | 2 | 173 | AGAGGCGGCTATGCGGGTAC | 7 | 15 |
| 178173 | 2 | 196 | GGGCAGCCCGAAGGCCTGGT | 18 | 16 |
| 178182 | 2 | 229 | GCCTAACGACTGCGACCACT | 75 | 17 |
| 178150 | 2 | 244 | TGGCCAGCTGCTGCCGCCTA | 0 | 18 |
| 178166 | 2 | 245 | CTGGCCAGCTGCTGCCGCCT | 0 | 19 |
| 178161 | 2 | 333 | CTGAGTTGCCGGCTGAGCGC | 0 | 20 |
| 178163 | 2 | 348 | TCCGAGACCCCGCTGCTGAG | 39 | 21 |
| 178175 | 2 | 352 | GATCTCCGAGACCCCGCTGC | 37 | 22 |
| 178152 | 2 | 389 | CATCCAGGGACACGCGCCAG | 0 | 23 |
| 178177 | 2 | 426 | GTCTTGACCGTCAGCTCGTC | 83 | 24 |
| 178179 | 2 | 455 | TGCCGGTGATCTCCACCACG | 18 | 25 |
| 178186 | 2 | 458 | GCTTGCCGGTGATCTCCACC | 0 | 26 |
| 178145 | 2 | 464 | CCTCGTGCTTGCCGGTGATC | 29 | 27 |
| 178143 | 2 | 481 | ATGCTCGTCCTGCCGCTCCT | 49 | 28 |
| 178191 | 2 | 484 | GCCATGCTCGTCCTGCCGCT | 56 | 29 |
| 178141 | 2 | 489 | ATGTAGCCATGCTCGTCCTG | 59 | 30 |
| 178195 | 2 | 490 | GATGTAGCCATGCTCGTCCT | 64 | 31 |
| 178200 | 2 | 515 | TGTATTTCCGCGTGAAGCAC | 40 | 32 |
| 178168 | 2 | 588 | GCCTCCAGGGTCAGTGTGCC | 0 | 33 |
| 178165 | 2 | 618 | TTGGACTGCGTGGCTAGCTT | 59 | 34 |
| 178198 | 2 | 621 | TCGTTGGACTGCGTGGCTAG | 52 | 35 |
| 178172 | 2 | 625 | GATCTCGTTGGACTGCGTGG | 36 | 36 |
| 178170 | 2 | 628 | GGTGATCTCGTTGGACTGCG | 43 | 37 |
| 178202 | 2 | 641 | AGGTGACTGGGATGGTGATC | 47 | 38 |
| 178147 | 2 | 675 | GCTTCTGGGCCCCCAAGCTG | 13 | 39 |
| 178189 | 2 | 697 | GGCAGTCTCATCGGATTTTG | 67 | 40 |
| 178204 | 2 | 711 | AGGCTTTACTTGGCGGCAGT | 11 | 41 |
| 178188 | 2 | 718 | CAGGCTAAGGCTTTACTTGG | 36 | 42 |

As shown in Table 3, SEQ ID NOs: 8, 10, 13, 14, 16, 17, 21, 22, 24, 25, 27, 28, 29, 30, 31, 32, 34, 35, 36, 37, 38, 40 and 42 inhibited expression of HSP27 by at least 18%; SEQ ID NOs: 13, 14, 17, 21, 22, 24, 28, 29, 30, 31, 32, 34, 35, 36, 37, 38, 40 and 42 inhibited expression of HSP27 by at least 36%; and SEQ ID NOs: 17, 24, 29, 30, 31, 34, 35 and 40 inhibited expression of HSP27 by at least 52%. In one embodiment, antisense compounds targeting nucleotides 229-248 of SEQ ID NO: 2 are preferred. In another embodiment, antisense compounds targeting nucleotides 464-534, 481-534, 481-509 or 484-509 of SEQ ID NO: 2 are preferred. In another embodiment, antisense compounds targeting nucleotides 618-660, 618-640 or 618-637 of SEQ ID NO: 2 are preferred. In another embodiment, antisense compounds targeting nucleotides 697-716 of SEQ ID NO: 2 are preferred.

1. An antisense compound 15 to 30 nucleobases in length targeted to nucleotides 618 to 660 of a nucleic acid molecule encoding human HSP27 (SEQ ID NO: 2).

2. The compound of claim 1 targeted to nucleotides 618 to 640 of SEQ ID NO: 2.

3. The compound of claim 1 targeted to nucleotides 618 to 637 of SEQ ID NO: 2.

4. An antisense compound 15 to 30 nucleobases in length targeted to nucleotides 229 to 248 of a nucleic acid molecule encoding human HSP27 (SEQ ID NO: 2).

5. An antisense compound 15 to 30 nucleobases in length targeted to nucleotides 697 to 716 of a nucleic acid molecule encoding human HSP27 (SEQ ID NO: 2).

6. An antisense compound 15 to 30 nucleobases in length targeted to nucleotides 464 to 534 of a nucleic acid molecule encoding human HSP27 (SEQ ID NO: 2).

7. The compound of claim 6 targeted to nucleotides 481 to 534 of SEQ ID NO: 2.

8. The compound of claim 6 targeted to nucleotides 481 to 509 of SEQ ID NO: 2.

9. The compound of claim 6 targeted to nucleotides 484 to 534 of SEQ ID NO: 2.

10. The compound of any one of the preceding claims comprising at least one modified internucleoside linkage, sugar moiety, or nucleobase.

11. The compound of claim 10 wherein said modified internucleoside linkage is a phosphorothioate.

12. The compound of claim 10 wherein said modified sugar moiety is 2′-O-methoxyethyl.

13. The compound of claim 10 wherein said modified nucleobase is 5-methylcytosine.

14. The compound of claim 10 which is a chimeric oligonucleotide.

15. A pharmaceutical composition comprising the compound of claim 1 and a pharmaceutically acceptable penetration enhancer, carrier, or diluent.

16. A method of inhibiting the expression of HSP27 in a cell, tissue or animal comprising contacting said cell, tissue or animal with the compound claim 1.


SEQUENCE LISTING (42 sequences)

SEQ ID NO: 1 - DNA, 764 bp, Homo sapiens
ggcacgaggagcagagtcagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaaagccttagcccggatgcccacccctgctgccgccactggctgtgcctcccccgccacctgtgtgttcttttgatacatttatcttctgtttttctcaaataaagttcaaagcaaccacctg

SEQ ID NO: 2 - DNA, 865 bp, Homo sapiens
ctcaaacaccgcctgctaaaaatacccgactggaggagcataaaagcgcagccgagcccagcgccccgcacttttctgagcagacgtccagagcagagtcagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgaggagcggcaggacgagcatggctacatctcccggtgcttcacgcggaaatacacgctgccccccggtgtggaccccacccaagtttcctcctccctgtcccctgagggcacactgaccgtggaggcccccatgcccaagctagccacgcagtccaacgagatcaccatcccagtcaccttcgagtcgcgggcccagcttgggggcccagaagctgcaaaatccgatgagactgccgccaagtaaagccttagcctggatgcccacccctgctgccgccactggctgtgcctcccccgccacctgtgtgttcttttgatacatttatcttctgtttttctcaaataaagttcaaagcaaccacctgtaaaaaaaaaaaaaaaaaa

SEQ ID NO: 3 - DNA, 21 bp, Artificial Sequence
  PCR Primer
tccctggatgtcaaccacttc

SEQ ID NO: 4 - DNA, 18 bp, Artificial Sequence
  PCR Primer
tctccaccacgccatcct

SEQ ID NO: 5 - DNA, 21 bp, Artificial Sequence
  Probe
cccggacgagctgacggtcaa

SEQ ID NO: 6 - DNA, 20 bp, Artificial Sequence
  Oligomeric Compound
ggtatttttagcaggcggtg

SEQ ID NO: 7 - DNA, 20 bp, Artificial Sequence
  Oligomeric Compound
ccagtcgggtatttttagca

SEQ ID NO: 8 - DNA, 20 bp, Artificial Sequence
  Oligomeric Compound
ctccagtcgggtatttttag

SEQ ID NO: 9 - DNA, 20 bp, Artificial Sequence
  Oligomeric Compound
tgctcctccagtcgggtatt

SEQ ID NO: 10 - DNA, 20 bp, Artificial Sequence
  Oligomeric Compound
cggctgcgcttttatgctcc

SEQ ID NO: 11 - DNA, 20 bp, Artificial Sequence
  Oligomeric Compound
gctcggctgcgcttttatgc

SEQ ID NO: 12 - DNA, 20 bp, Artificial Sequence
  Oligomeric Compound
ggctcggctgcgcttttatg

SEQ ID NO: 13 - DNA, 20 bp, Artificial Sequence
  Oligomeric Compound
atgctggctgactctgctct

SEQ ID NO: 14 - DNA, 20 bp, Artificial Sequence
  Oligomeric Compound
gaggcggctatgcgggtacc

SEQ ID NO: 15 - DNA, 20 bp, Artificial Sequence
  Oligomeric Compound
agaggcggctatgcgggtac

SEQ ID NO: 16 - DNA, 20 bp, Artificial Sequence
  Oligomeric Compound
gggcagcccgaaggcctggt

SEQ ID NO: 17 - DNA, 20 bp, Artificial Sequence
  Oligomeric Compound
gcctaaccactgcgaccact

SEQ ID NO: 18 - DNA, 20 bp, Artificial Sequence
  Oligomeric Compound
tggccagctgctgccgccta

SEQ ID NO: 19 - DNA, 20 bp, Artificial Sequence
  Oligomeric Compound
ctggccagctgctgccgcct

SEQ ID NO: 20 - DNA, 20 bp, Artificial Sequence
  Oligomeric Compound
ctgagttgccggctgagcgc

SEQ ID NO: 21 - DNA, 20 bp, Artificial Sequence
  Oligomeric Compound
tccgagaccccgctgctgag

SEQ ID NO: 22 - DNA, 20 bp, Artificial Sequence
  Oligomeric Compound
gatctccgagaccccgctgc

SEQ ID NO: 23 - DNA, 20 bp, Artificial Sequence
  Oligomeric Compound
catccagggacacgcgccag

SEQ ID NO: 24 - DNA, 20 bp, Artificial Sequence
  Oligomeric Compound
gtcttgaccgtcagctcgtc

SEQ ID NO: 25 - DNA, 20 bp, Artificial Sequence
  Oligomeric Compound
tgccggtgatctccaccacg

SEQ ID NO: 26 - DNA, 20 bp, Artificial Sequence
  Oligomeric Compound
gcttgccggtgatctccacc

SEQ ID NO: 27 - DNA, 20 bp, Artificial Sequence
  Oligomeric Compound
cctcgtgcttgccggtgatc

SEQ ID NO: 28 - DNA, 20 bp, Artificial Sequence
  Oligomeric Compound
atgctcgtcctgccgctcct

SEQ ID NO: 29 - DNA, 20 bp, Artificial Sequence
  Oligomeric Compound
gccatgctcgtcctgccgct

SEQ ID NO: 30 - DNA, 20 bp, Artificial Sequence
  Oligomeric Compound
atgtagccatgctcgtcctg

SEQ ID NO: 31 - DNA, 20 bp, Artificial Sequence
  Oligomeric Compound
gatgtagccatgctcgtcct

SEQ ID NO: 32 - DNA, 20 bp, Artificial Sequence
  Oligomeric Compound
tgtatttccgcgtgaagcac

SEQ ID NO: 33 - DNA, 20 bp, Artificial Sequence
  Oligomeric Compound
gcctccacggtcagtgtgcc

SEQ ID NO: 34 - DNA, 20 bp, Artificial Sequence
  Oligomeric Compound
ttggactgcgtggctagctt

SEQ ID NO: 35 - DNA, 20 bp, Artificial Sequence
  Oligomeric Compound
tcgttggactgcgtggctag

SEQ ID NO: 36 - DNA, 20 bp, Artificial Sequence
  Oligomeric Compound
gatctcgttggactgcgtgg

SEQ ID NO: 37 - DNA, 20 bp, Artificial Sequence
  Oligomeric Compound
ggtgatctcgttggactgcg

SEQ ID NO: 38 - DNA, 20 bp, Artificial Sequence
  Oligomeric Compound
aggtgactgggatggtgatc

SEQ ID NO: 39 - DNA, 20 bp, Artificial Sequence
  Oligomeric Compound
gcttctgggcccccaagctg

SEQ ID NO: 40 - DNA, 20 bp, Artificial Sequence
  Oligomeric Compound
ggcagtctcatcggattttg

SEQ ID NO: 41 - DNA, 20 bp, Artificial Sequence
  Oligomeric Compound
aggctttacttggcggcagt

SEQ ID NO: 42 - DNA, 20 bp, Artificial Sequence
  Oligomeric Compound
caggctaaggctttacttgg